SEQUENCE LISTING (10 sequences)

SEQ ID NO: 1 - DNA, 1909 bp, Homo sapiens
gacaccttttaaaatgcagaactaactgaggcatttcagtaactttgctttcaaatcaataaagtcaaatgtatggaaacattttgtgccctactctccataccctgtgtactcaaattctctactgtatgaattatgctttaagtagaattcagtgccaaggagaacttggtgaaataaattattttaattttttttttatcctttacaaagccatggattttatttggttgatgtgtgctctgtacacaagccatttcaataggatggagctgttaattattttccaaagagtaatagacatgcaaaagtttcaataaaaactgggccattaacaaataaattaataaactaataagcattcccttctaggtttttgccaaactgcctatccaataacaaatttgagaatcgttgaaaaagctagttatatttcagagaaatgattttcattattgaaactgttctccctagcaggccattttccctttttcctgggagtttagcaagtttaggagagaatagtcatgaaaagaaagggaagaaaggggagaagggaagaggttaaaaagtaagtgctcagacctatgaacgtaatccctttgctagaaatatttaagagcagctcagcttggttgaaactgagttttgtcatcttccatatttgcaggaaggtattttctgacttgcaatgcagctagatgtaaaattttattttatcatcctagaaagccttgactagaaaaatgaataaatattgagggtttcctgtccatatctggcttgcatgtgccagaaagcagagaatagaaaatgtaatctccaacatccaagcatcgaaacccaaggggtaggcaattctatgtaggttttggacatgaagtttggtgcatcttggtttatgctggctcaactgctattaaacctctctggcttatagtctcttcattctattagacaagcacgtatcgaacacttgcttcgcacaaggctctttagttaacaatttagcagctactgtttgtgttaaacacacttttcaccaaataggttctgaggcaaacgagagcaatgactatttaaagaaaggctttcccagcatcacttacacatcccaaaactaaaaagatcaactcttccaactgagaaaagactcctggctttgaatggaaacttacagcagagagtcacaggccacggcaacaacaacgacaacaacaaacatttggaatattattctcaactcacgttttaataatacatcttaattatttttctagtagagaaactacaaatcagcctcttcaacatttatatacagtttaataagcctcttgcaagttacttgttctctcacctgaggtatttttttcctccccaccttgcccctgttcctcccttcctcttctccctttgcaagaggaaatatttaacatatttgggtccaacttcaataatgtaataattaatacattaaaagcatttaacttcctttctagaaaaatgcacaggctaaggcatagacaaaacaaagagaaatgctgagaaatttgccactggagacaagcaatctgaataaatatttgccaaaagttctttttatgtcatatagtgtcaggatttgaaggagctattttttttaatgttgcaactagcaactcatcttcggaagacacagccaggagaatgaagtagaagtgaaaggtttataaatccatttgtaagcatttatcccatatattttaaattcaagaaaaattgtgtttatctttagaattttgtattcaatactttatgtactatgtgactcatgcttctggataaataaagcaccaaatatgtatctgtaaccacaatcacacatattatattaaatatatatctatataacaaaaaaaaaaaaaaaaaa

SEQ ID NO: 2 - protein, 83 aa, Homo sapiens
MYGNILCPTLHTLCTQILYCMNYALSRIQCQGELGEINYFNFFFILYKAMDFIWLMCALYTSHFNRMELLIIFQRVIDMQKFQ

SEQ ID NO: 3 - DNA, 2064 bp, Homo sapiens
tgcaagtctgcagccagcagagctcacagttgttgcaaagtgctcagcactaagggagccagcgcacagcacagccaggaaggcgagcgagcccagccagcccagccagcccagccagcccggaggtcatttgattgcccgcctcagaacgatggatctgcatctcttcgactactcagagccagggaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcggcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggctgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactctgccttggagcagagcaccaaatgatctgccctggagaggctctgggacgggtttacttgtttttgaacagggtgatgggccctatggttttctagagcaaagcaaagtagcttcgggtcttgatgcttgagtagagtgaagaggggagcacgtgccccctgcatccattctctctttctcttgatgacgcagctgtcatttggctgtgcgtgctgacagttttgcaacaggcagagctgtgtcgcacagcagtgctgtgcgtcagagccagctgaggacaggcttgcctggacttctgtaagataggattttctgtgtttcctgaattttttatatggtgatttgtatttaaattttaagactttattttctcactattggtgtaccttataaatgtatttgaaagttaaatatattttaaatattgtttgggaggcatagtgctgacatatattcagagtgttgtagttttaaggttagcgtgacttcagttttgactaaggatgacactaattgttagctgttttgaaattatatatatataaatatatataaatatataaatatatgccagtcttggctgaaatgttttatttaccatagttttatatctgtgtggtgttttgtaccggcacgggatatggaacgaaaactgctttgtaatgcagtttgtgacattaatagtattgtaaagttacattttaaaataaacaaaaaactgttctggactgcaaatctgcacacacaacgaacagttgcatttcagagagttctctcaatttgtaagttatttttttttaataaagatttttgtttccaaaaaaaaaaaaaaaaaaaaaaaaaaaaaa

SEQ ID NO: 4 - protein, 362 aa, Homo sapiens
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 5 - DNA, 4286 bp, Homo sapiens
gagacattccggtgggggactctggccagcccgagcaacgtggatcctgagagcactcccaggtaggcatttgccccggtgggacgccttgccagagcagtgtgtggcaggcccccgtggaggatcaacacagtggctgaacactgggaaggaactggtacttggagtctggacatctgaaacttggctctgaaactgcgcagcggccaccggacgccttctggagcaggtagcagcatgcagccgcctccaagtctgtgcggacgcgccctggttgcgctggttcttgcctgcggcctgtcgcggatctggggagaggagagaggcttcccgcctgacagggccactccgcttttgcaaaccgcagagataatgacgccacccactaagaccttatggcccaagggttccaacgccagtctggcgcggtcgttggcacctgcggaggtgcctaaaggagacaggacggcaggatctccgccacgcaccatctcccctcccccgtgccaaggacccatcgagatcaaggagactttcaaatacatcaacacggttgtgtcctgccttgtgttcgtgctggggatcatcgggaactccacacttctgagaattatctacaagaacaagtgcatgcgaaacggtcccaatatcttgatcgccagcttggctctgggagacctgctgcacatcgtcattgacatccctatcaatgtctacaagctgctggcagaggactggccatttggagctgagatgtgtaagctggtgcctttcatacagaaagcctccgtgggaatcactgtgctgagtctatgtgctctgagtattgacagatatcgagctgttgcttcttggagtagaattaaaggaattggggttccaaaatggacagcagtagaaattgttttgatttgggtggtctctgtggttctggctgtccctgaagccataggttttgatataattacgatggactacaaaggaagttatctgcgaatctgcttgcttcatcccgttcagaagacagctttcatgcagttttacaagacagcaaaagattggtggctgttcagtttctatttctgcttgccattggccatcactgcatttttttatacactaatgacctgtgaaatgttgagaaagaaaagtggcatgcagattgctttaaatgatcacctaaagcagagacgggaagtggccaaaaccgtcttttgcctggtccttgtctttgccctctgctggcttccccttcacctcagcaggattctgaagctcactctttataatcagaatgatcccaatagatgtgaacttttgagctttctgttggtattggactatattggtatcaacatggcttcactgaattcctgcattaacccaattgctctgtatttggtgagcaaaagattcaaaaactgctttaagtcatgcttatgctgctggtgccagtcatttgaagaaaaacagtccttggaggaaaagcagtcgtgcttaaagttcaaagctaatgatcacggatatgacaacttccgttccagtaataaatacagctcatcttgaaagaagaactattcactgtatttcattttctttatattggaccgaagtcattaaaacaaaatgaaacatttgccaaaacaaaacaaaaaactatgtatttgcacagcacactattaaaatattaagtgtaattattttaacactcacagctacatatgacattttatgagctgtttacggcatggaaagaaaatcagtgggaattaagaaagcctcgtcgtgaaagcacttaattttttacagttagcacttcaacatagctcttaacaacttccaggatattcacacaacacttaggcttaaaaatgagctcactcagaatttctattctttctaaaaagagatttatttttaaatcaatgggactctgatataaaggaagaataagtcactgtaaaacagaacttttaaatgaagcttaaattactcaatttaaaattttaaaatcctttaaaacaacttttcaattaatattatcacactattatcagattgtaattagatgcaaatgagagagcagtttagttgttgcatttttcggacactggaaacatttaaatgatcaggagggagtaacagaaagagcaaggctgtttttgaaaatcattacactttcactagaagcccaaacctcagcattctgcaatatgtaaccaacatgtcacaaacaagcagcatgtaacagactggcacatgtgccagctgaatttaaaatataatacttttaaaaagaaaattattacatcctttacattcagttaagatcaaacctcacaaagagaaatagaatgtttgaaaggctatcccaaaagacttttttgaatctgtcattcacataccctgtgaagacaatactatctacaattttttcaggattattaaaatcttcttttttcactatcgtagcttaaactctgtttggttttgtcatctgtaaatacttacctacatacactgcatgtagatgattaaatgagggcaggccctgtgctcatagctttacgatggagagatgccagtgacctcataataaagactgtgaactgcctggtgcagtgtccacatgacaaaggggcaggtagcaccctctctcacccatgctgtggttaaaatggtttctagcatatgtataatgctatagttaaaatactatttttcaaaatcatacagattagtacatttaacagctacctgtaaagcttattactaatttttgtattatttttgtaaatagccaatagaaaagtttgcttgacatggtgcttttctttcatctagaggcaaaactgctttttgagaccgtaagaacctcttagctttgtgcgttcctgcctaatttttatatcttctaagcaaagtgccttaggatagcttgggatgagatgtgtgtgaaagtatgtacaagagaaaacggaagagagaggaaatgaggtggggttggaggaaacccatggggacagattcccattcttagcctaacgttcgtcattgcctcgtcacatcaatgcaaaaggtcctgattttgttccagcaaaacacagtgcaatgttctcagagtgactttcgaaataaattgggcccaagagctttaactcggtcttaaaatatgcccaaatttttactttgtttttcttttaataggctgggccacatgttggaaataagctagtaatgttgttttctgtcaatattgaatgtgatggtacagtaaaccaaaacccaacaatgtggccagaaagaaagagcaataataattaattcacacaccatatggattctatttataaatcacccacaaacttgttctttaatttcatcccaatcactttttcagaggcctgttatcatagaagtcattttagactctcaattttaaattaattttgaatcactaatattttcacagtttattaatatatttaatttctatttaaattttagattatttttattaccatgtactgaatttttacatcctgataccctttccttctccatgtcagtatcatgttctctaattatcttgccaaattttgaaactacacacaaaaagcatacttgcattatttataataaaattgcattcagtggctttttaaaaaaaatgtttgattcaaaactttaacatactgataagtaagaaacaattataatttctttacatactcaaaaccaagatagaaaaaggtgctatcgttcaacttcaaaacatgtttcctagtattaaggactttaatatagcaacagacaaaattattgttaacatggatgttacagctcaaaagatttataaaagattttaacctattttctcccttattatccactgctaatgtggatgtatgttcaaacaccttttagtattgatagcttacatatggccaaaggaatacagtttatagcaaaacatgggtatgctgtagctaactttataaaagtgtaatataacaatgtaaaaaattatatatctgggaggattttttggttgcctaaagtggctatagttactgattttttattatgtaagcaaaaccaataaaaatttaagtttttttaacaactaccttatttttcactgtacagacactaattcattaaatactaattgattgtttaaaagaaatataaatgtgacaagtggacattatttatgttaaatatacaattatcaagcaagtatgaagttattcaattaaaatgccacatttctggtctctggg

SEQ ID NO: 6 - protein, 436 aa, Homo sapiens
MQPPPSLCGRALVALVLACGLSRIWGEERGFPPDRATPLLQTAEIMTPPTKTLWPKGSNASLARSLAPAEVPKGDRTAGSPPRTISPPPCQGPIEIKETFKYINTVVSCLVFVLGIIGNSTLLRIIYKNKCMRNGPNILIASLALGDLLHIVIDIPINVYKLLAEDWPFGAEMCKLVPFIQKASVGITVLSLCALSIDRYRAVASWSRIKGIGVPKWTAVEIVLIWVVSVVLAVPEAIGFDIITMDYKGSYLRICLLHPVQKTAFMQFYKTAKDWWLFSFYFCLPLAITAFFYTLMTCEMLRKKSGMQIALNDHLKQRREVAKTVFCLVLVFALCWLPLHLSRILKLTLYNQNDPNRCELLSFLLVLDYIGINMASLNSCINPIALYLVSKRFKNCFKAGPHVGNKLVMLFSVNIECDGTVNQNPTMWPERKSNNN

SEQ ID NO: 7 - DNA, 8091 bp, Homo sapiens
acgcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcgccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggtgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgtgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccgggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccggaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcaggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggtggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttctttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcctcctccccaccccacctccctcacttcactgcattccagattggacatgttccatagccttgctggggaagggcccactgccaactccctctgccccagccccacccttggccatctccctttgggaactagggggctgctggtgggaaatgggagccagggcagatgtatgcattcctttatgtccctgtaaatgtgggactacaagaagaggagctgcctgagtggtactttctcttcctggtaatcctctggcccagccttatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaaaa

SEQ ID NO: 8 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 9 - DNA, 1638 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaaaaaaaaaaaaaaa

SEQ ID NO: 10 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

(19) United States
(12) Patent Application Publication
(10) Pub. No.: US 2011/0104059 A1
(43) Pub. Date: May 5, 2011

(54) Membrane Associated Tumor Endothelium Markers

(75) Inventors: Brad ST. CROIX, Frederick, MD (US); Kenneth W. KINZLER, Bel Air, MD (US); Bert VOGELSTEIN, Baltimore, MD (US)

(73) Assignee: THE JOHNS HOPKINS UNIVERSITY, Baltimore, MD (US)

(21) Appl. No.: 12/436,444

(22) Filed: May 6, 2009

Related U.S. Application Data
(63) Continuation of application No. 10/518,751, filed on Jun. 19, 2006, now abandoned.
(60) Provisional application No. 60/390,187, filed on Jun. 21, 2002.
(60) Provisional application No. 60/458,959, filed on Apr. 1, 2003.

(51) Int. Cl. A61K 49/00; C07K 16/24; C07K 16/18; C07K 16/40; C07K 16/42; C07K 16/28; C07K 16/10; A61K 39/395; C07K 2/00; C07K 14/56; C07K 16/00; A61K 38/02; A61K 38/21; A61K 38/48; A61K 35/12; A61K 38/44; G01N 33/68; C12Q 1/68; A61K 39/21; A61K 39/00; A61P 35/00; A61P 19/02; A61P 17/06; A61P 27/02

(52) U.S. Cl. 424/9.1; 530/389.2; 530/389.7; 530/389.8; 530/387.2; 530/389.4; 530/387.3; 530/391.7; 530/391.3; 424/172.1; 424/131.1; 424/158.1; 424/174.1; 424/159.1; 530/350; 530/351; 530/389.1; 514/13.3; 424/85.7; 424/94.67; 424/130.1; 424/93.7; 424/94.4; 436/501; 435/7.1; 435/6; 424/207.1; 424/277.1; 424/184.1

(57) ABSTRACT

To gain a better understanding of tumor angiogenesis endothelial cells (ECs) were isolated and gene expression patterns were evaluated. When transcripts from ECs derived from normal and malignant colorectal tissues were compared with transcripts from non-endothelial cells, over 170 genes predominantly expressed in the endothelium were identified. Comparison between normal- and tumor-derived endothelium revealed differentially expressed genes, including many that were specifically elevated in tumor-associated endothelium. Experiments with representative genes from this group demonstrated that most were similarly expressed in the endothelium of primary lung, breast, brain, and pancreatic cancers as well as in metastatic lesions of the liver. These results demonstrate that neoplastic and normal endothelium in humans are distinct at the molecular level.

The U.S. government retains certain rights in the invention by virtue of the provisions of National Institutes of Health grants CA57345 and CA43460, which support this work.

This Application incorporates by reference the contents of each of two duplicate CD-ROMs. Each CD-ROM contains an identical 53.5 kB file labeled “00358jhu.seq sequence listing.txt and containing the sequence listing for this application. The CD-ROMs were created on Dec. 17, 2004.

### TECHNICAL FIELD OF THE INVENTION

This invention is related to the area of angiogenesis and anti-angiogenesis. In particular, it relates to genes which are characteristically expressed in tumor endothelial and normal endothelial cells.

### BACKGROUND OF THE INVENTION

It is now widely recognized that tumors require a blood supply for expansive growth. This recognition has stimulated a profusion of research on tumor angiogenesis, based on the idea that the vasculature in tumors represents a potential therapeutic target. However, several basic questions about tumor endothelium remain unanswered. For example, are vessels of tumors qualitatively different from normal vessels of the same tissue? What is the relationship of tumor endothelium to endothelium of healing wounds or other physiological or pathological forms of angiogenesis? The answers to these questions critically impact on the potential for new therapeutic approaches to inhibit angiogenesis in a specific manner.

There is a continuing need in the art to characterize the vasculature of tumors relative to normal vasculature so that any differences can be exploited for therapeutic and diagnostic benefits.

One technique which can be used to characterize gene expression, or more precisely gene transcription, is termed serial analysis of gene expression (SAGE). Briefly, the SAGE approach is a method for the rapid quantitative and qualitative analysis of mRNA transcripts based upon the isolation and analysis of short defined sequence tags (SAGE Tags) corresponding to expressed genes. Each Tag is a short nucleotide sequences (9-17 base pairs in length) from a defined position in the transcript. In the SAGE method, the Tags are dimerized to reduce bias inherent in cloning or amplification reactions. (See, U.S. Pat. No. 5,695,937.) SAGE is particularly suited to the characterization of genes associated with vasculature stimulation or inhibition because it is capable of detecting rare sequences, evaluating large numbers of sequences at one time, and to provide a basis for the identification of previously unknown genes.

### SUMMARY OF THE INVENTION

One embodiment of the invention provides an isolated molecule comprising an antibody variable region which specifically binds to an extracellular domain of a TEM protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D021 1; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. The molecule can be, for example, an intact antibody molecule, a single chain variable region (ScFv), a monoclonal antibody, a humanized antibody, or a human antibody. The molecule can optionally be bound to a cytotoxic moiety, bound to a therapeutic moiety, bound to a detectable moiety, or bound to an anti-tumor agent.

According to another embodiment of the invention a method of inhibiting neoangiogenesis is provided. An effective amount of an isolated molecule comprising an antibody variable region which specifically binds to an extracellular domain of a TEM protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D021 1; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1, is administered to a subject in need thereof . Neoangiogenesis is consequently inhibited. The subject may bear a vascularized tumor, may have polycystic kidney disease, may have diabetic retinopathy, may have rheumatoid arthritis, may have psoriasis, for example.

Another aspect of the invention is a method of inhibiting tumor growth. An effective amount of an isolated molecule comprising an antibody variable region which specifically binds to an extracellular domain of a TEM protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1, is administered to a human subject bearing a tumor. The growth of the tumor is consequently inhibited.

Another aspect of the invention is a method for identification of a ligand involved in endothelial cell regulation. A test compound is contacted with an isolated and purified human trasmembrane protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. The isolated and purified human trasmembrane protein is also contacted with a molecule comprising an antibody variable region which specifically binds to an extracellular domain of a TEM protein selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. Binding of the molecule comprising an antibody variable region to the human transmembrane protein is determined. A test compound which diminishes the binding of the molecule comprising an antibody variable region to the human transmembrane protein is identified as a ligand involved in endothelial cell regulation. The test compound can be further tested to determine its effect on endothelial cell growth, either in culture or in a mammal.

Yet another aspect of the invention is a method for identification of a ligand involved in endothelial cell regulation. A test compound is contacted with a cell comprising a human transmembrane protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. The cell is also contacted with a molecule comprising an antibody variable region which specifically binds to an extracellular domain of a protein selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D021 1; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. Binding of the molecule comprising an antibody variable region to the cell is determined. A test compound that diminishes the binding of the molecule comprising an antibody variable region to the cell is identified as a ligand involved in endothelial cell regulation. The test compound can be further tested to determine its effect on endothelial cell growth, either in culture or in a mammal.

Yet another aspect of the invention is a method for identification of a ligand involved in endothelial cell regulation. A test compound is contacted with a human transmembrane protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. Binding of a test compound to the human transmembrane protein is determined. A test compound which binds to the protein is identified as a ligand involved in endothelial cell regulation. The test compound can be further tested to determine its effect on endothelial cell growth, either in culture or in a mammal.

Another embodiment of the present invention is a soluble form of a human transmembrane protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. The soluble forms lack transmembrane domains. The soluble form may consist of an extracellular domain of the human transmembrane protein.

Also provided by the present invention is a method of inhibiting neoangiogenesis in a patient. A soluble form of a human transmembrane protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1 is adminstered to the patient. Neoangiogenesis in the patient is consequently inhibited. The patient may bear a vascularized tumor, may have polycystic kidney disease, may have diabetic retinopathy, may have rheumatoid arthritis, or may have psoriasis, for example.

According to still another aspect of the invention a method of identifying regions of neoangiogenesis in a patient is provided. A molecule comprising an antibody variable region which specifically binds to an extracellular domain of a TEM protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1, is administered to a patient. The molecule is bound to a detectable moiety. The detectable moiety is detected in the pateint, thereby identifying neoangiogenesis.

Still another embodiment of the invention is a method of screening for neoangiogenesis in a patient. A body fluid collected from the patient is contacted with a molecule comprising an antibody variable region which specifically binds to an extracellular domain of a protein selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. Detection of cross-reactive material in the body fluid with the molecule indicates neo-angiogenesis in the patient.

A still further embodiment of the invention is a method to identify candidate drugs for treating tumors. Cells which express one or more genes selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1 respectively, are contacted with a test compound. Expression of said one or more genes is determined by hybridization of mRNA of said cells to a nucleic acid probe which is complementary to an mRNA of said one or more genes. A test compound is identified as a candidate drug for treating tumors if it decreases expression of said one or more genes. Optionally the cells are endothelial cells. Alternatively or additionally, the cells are recombinant host cells which are transfected with an expression construct for said one or more genes. Test compounds that increase expression can be identified as candidates for promoting wound healing.

Yet another embodiment of the invention is a method to identify candidate drugs for treating tumors. Cells which express one or more proteins selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1, are contacted with a test compound. The amount of said one or more of said proteins in said cells is determined. A test compound is identified as a candidate drug for treating tumors if it decreases the amount of one or more of said proteins in said cells. Optionally the cells are endothelial cells. Alternatively or additionally, the cells are recombinant host cells which are transfected with an expression construct which encodes said one or more proteins. Alternatively, a test compound that increases the amount of one or more of said proteins in said cells is identified as a candidate drug for treating wound healing.

According to another aspect of the invention a method is provided to identify candidate drugs for treating tumors. Cells which express one or more proteins selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1, are contacted with a test compound. Activity of said one or more proteins in said cells is determined. A test compound is identified as a candidate drug for treating tumors if it decreases the activity of one more of said proteins in said cells. Optionally the cells are endothelial cells. Alternatively or additionally, the cells are recombinant host cells which are transfected with an expression construct which encodes said one or more proteins. Optionally the cells are endothelial cells. If a test compound increases the acitivity of one more of said proteins in said cells it can be identified as a candidate drug for treating wound healing.

An additional aspect of the invention is a method to identify candidate drugs for treating patients bearing tumors. A test compound is contacted with recombinant host cells which are transfected with an expession construct which encodes one or more proteins selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D021 1; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. Proliferation of said cells is determined. A test compound which inhibits proliferation of said cells is identified as a candidate drug for treating patients bearing tumors. A test coumpound which stimulates proliferation of said cells is identified as a candidate drug for promoting neoangiogenesis, such as for use in wound healing.

Another aspect of the invention is a method for identifying endothelial cells. One or more molecules comprising a variable region which binds specifically to a protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D021 1; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1 is contacted with a population of cells. Cells in the population which have bound to said molecules are detected. Cells which are bound to said molecules are identified as endothelial cells. Optionally cells which have bound to said molecules are isolated from cells which have not bound. Such molecules can be intact antibodies, for example.

Still another aspect of the invention is a method for identifying endothelial cells. One or more nucleic acid hybridization probes which are complementary to a DNA, cDNA, or mRNA identified by a nucleic acid sequence selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1 is contacted with cDNA or mRNA of a population of cells. cDNA or mRNA which have specifically hybridized to said nucleic acid hybridization probes are detected. Cells whose cDNA or mRNA specifically hybridized are identified as endothelial cells.

In another embodiment of the invention a method is provided for inducing an immune response to a TEM protein in a mammal. Such immunity can be used to prevent, arrest, or inhibit spread of tumor cells in the body. A TEM protein or a nucleic acid encoding a TEM protein is administered to a human subject who has or is at risk of developing a tumor. The TEM protein is protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. A humoral or cellular immune response to the TEM protein is thereby raised in the human subject. Immune adjuvants can be used to augment the immune response.

According to another embodiment of the invention vascular proliferation is stimulated by providing a TEM protein or nucleic acid endcoding a TEM protein to a subject in need thereof. The TEM protein is selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; CRELD1 cysteine-rich with EGF-like domains 1. Subjects in need of vascular proliferation are those with wounds, for example.

These and other embodiments which will be apparent to those of skill in the art upon reading the specification provide the art with reagents and methods for detection, diagnosis, therapy, and drug screening pertaining to neoangiogenesis and pathological processes involving or requiring neoangiogenesis.

### DETAILED DESCRIPTION OF THE INVENTION

We identified 76 human genes that are expressed at significantly higher levels (≧2-fold) in tumor endothelium than in normal endothelium and that encode membrane proteins. See Table 1. Most of these genes were either not expressed or expressed at relatively low levels in Endothelial Cells (ECs) maintained in culture. Interestingly, the tumor endothelium genes were expressed in all tumors tested, regardless of its tissue or organ source. Most tumor endothelium genes were also expressed in corpus luteum and wounds.

It is clear that normal and tumor endothelium are highly related, sharing many endothelial cell specific markers. It is equally clear that the endothelium derived from tumors is qualitatively different from that derived from normal tissues of the same type and is also different from primary endothelial cultures. These genes are characteristically expressed in tumors derived from several different tissue types, documenting that tumor endothelium, in general, is different from normal endothelium. The genes expressed differentially in tumor endothelium are also expressed during other angiogenic processes such as corpus luteum formation and wound healing. It is therefore more appropriate to regard the formation of new vessels in tumors as “neoangiogenesis” rather than “tumor angiogenesis” per se. This distinction is important from a variety of perspectives, and is consistent with the idea that tumors recruit vasculature using much of, or basically the same signals elaborated during other physiologic or pathological processes. That tumors represent “unhealed wounds” is one of the oldest ideas in cancer biology.

Sequence and literature study has permitted the following identifications to be made among the family of TEM proteins. Membrane associated TEM proteins have been identified which contain transmembrane regions. These include potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1.

ECs represent only a minor fraction of the total cells within normal or tumor tissues, and only those EC transcripts expressed at the highest levels would be expected to be represented in libraries constructed from unfractionated tissues. The genes described in the current study should therefore provide a valuable resource for basic and clinical studies of human angiogenesis in the future. Nucleic acids and/or proteins corresponding to each of these genes are identified in Unigene, OMIM, and/or protein databases as indicated in Table 1.

Isolated and purified nucleic acids, according to the present invention are those which are not linked to those genes to which they are linked in the human genome. Moreover, they are not present in a mixture such as a library containing a multitude of distinct sequences from distinct genes. They may be, however, linked to other genes such as vector sequences or sequences of other genes to which they are not naturally adjacent. Tags disclosed herein, because of the way that they were made, represent sequences which are 3′ of the 3′ most restriction enzyme recognition site for the tagging enzyme used to generate the SAGE tags. In this case, the tags are 3′ of the most 3′ most NlaIII site in the cDNA molecules corresponding to mRNA. Nucleic acids corresponding to tags may be RNA, cDNA, or genomic DNA, for example. Such corresponding nucleic acids can be determined by comparison to sequence databases to determine sequence identities. Sequence comparisons can be done using any available technique, such as BLAST, available from the National Library of Medicine, National Center for Biotechnology Information. Tags can also be used as hybridization probes to libraries of genomic or cDNA to identify the genes from which they derive. Thus, using sequence comparisons or cloning, or combinations of these methods, one skilled in the art can obtain full-length nucleic acid sequences. Genes corresponding to tags will contain the sequence of the tag at the 3′ end of the coding sequence or of the 3′ untranslated region (UTR), 3′ of the 3′ most recognition site in the cDNA for the restriction endonuclease which was used to make the tags. The nucleic acids may represent either the sense or the anti-sense strand. Nucleic acids and proteins although disclosed herein with sequence particularity, may be derived from a single individual. Allelic variants which occur in the population of humans are included within the scope of such nucleic acids and proteins. Those of skill in the art are well able to identify allelic variants as being the same gene or protein. Given a nucleic acid, one of ordinary skill in the art can readily determine an open reading frame present, and consequently the sequence of a polypeptide encoded by the open reading frame and, using techniques well known in the art, express such protein in a suitable host. Proteins comprising such polypeptides can be the naturally occurring proteins, fusion proteins comprising exogenous sequences from other genes from humans or other species, epitope tagged polypeptides, etc. Isolated and purified proteins are not in a cell, and are separated from the normal cellular constituents, such as nucleic acids, lipids, etc. Typically the protein is purified to such an extent that it comprises the predominant species of protein in the composition, such as greater than 50, 60 70, 80, 90, or even 95% of the proteins present.

Using the proteins according to the invention, one of ordinary skill in the art can readily generate antibodies which specifically bind to the proteins. Such antibodies can be monoclonal or polyclonal. They can be chimeric, humanized, or totally human. Any functional fragment or derivative of an antibody can be used including Fab, Fab', Fab2, Fab'2, and single chain variable regions. So long as the fragment or derivative retains specificity of binding for the endothelial marker protein it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific.

Techniques for making such partially to fully human antibodies are known in the art and any such techniques can be used. According to one particularly preferred embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. See for example, Nina D. Russel, Jose R. F. Corvalan, Michael L. Gallo, C. Geoffrey Davis, Liise-Anne Pirofski. Production of Protective Human Antipneumococcal Antibodies by Transgenic Mice with Human Immunoglobulin Loci Infection and Immunity April 2000, p. 1820-1826; Michael L. Gallo, Vladimir E. Ivanov, Aya Jakobovits, and C. Geoffrey Davis. The human immunoglobulin loci introduced into mice: V (D) and J gene segment usage similar to that of adult humans European Journal of Immunology 30: 534-540, 2000; Larry L. Green. Antibody engineering via genetic engineering of the mouse: XenoMouse strains are a vehicle for the facile generation of therapeutic human monoclonal antibodies Journal of Immunological Methods 231 11-23, 1999; Yang X-D, Corvalan J R F, Wang P, Roy C M-N and Davis C G. Fully Human Anti-interleukin-8 Monoclonal Antibodies: Potential Therapeutics for the Treatment of Inflammatory Disease States. Journal of Leukocyte Biology Vol. 66, pp 401-410 (1999); Yang X-D, Jia X-C, Corvalan J R F, Wang P, CG Davis and Jakobovits A. Eradication of Established Tumors by a Fully Human Monoclonal Antibody to the Epidermal Growth Factor Receptor without Concomitant Chemotherapy. Cancer Research Vol. 59, Number 6, pp 1236-1243 (1999) ; Jakobovits A. Production and selection of antigen-specific fully human monoclonal antibodies from mice engineered with human Ig loci. Advanced Drug Delivery Reviews Vol. 31, pp: 33-42 (1998); Green L and Jakobovits A. Regulation of B cell development by variable gene complexity in mice reconstituted with human immunoglobulin yeast artificial chromosomes. J. Exp. Med. Vol. 188, Number 3, pp: 483-495 (1998); Jakobovits A. The long-awaited magic bullets: therapeutic human monoclonal antibodies from transgenic mice. Exp. Opin. Invest. Drugs Vol. 7(4), pp : 607-614 (1998) ; Tsuda H, Maynard-Currie K, Reid L, Yoshida T, Edamura K, Maeda N, Smithies O, Jakobovits A. Inactivation of Mouse HPRT locus by a 203-bp retrotransposon insertion and a 55-kb gene-targeted deletion: establishment of new HPRT-Deficient mouse embryonic stem cell lines. Genomics Vol. 42, pp: 413-421 (1997); Sherman-Gold, R. Monoclonal Antibodies: The Evolution from '80s Magic Bullets To Mature, Mainstream Applications as Clinical Therapeutics. Genetic Engineering News Vol. 17, Number 14 (August 1997); Mendez M, Green L, Corvalan J, Jia X-C, Maynard-Currie C, Yang X-d, Gallo M, Louie D, Lee D, Erickson K, Luna J, Roy C, Abderrahim H, Kirschenbaum F, Noguchi M, Smith D, Fukushima A, Hales J, Finer M, Davis C, Zsebo K, Jakobovits A. Functional transplant of megabase human immunoglobulin loci recapitulates human antibody response in mice. Nature Genetics Vol. 15, pp: 146-156 (1997); Jakobovits A. Mice engineered with human immunoglobulin YACs: A new technology for production of fully human antibodies for autoimmunity therapy. Weir's Handbook of Experimental Immunology, The Integrated Immune System Vol. IV, pp: 194.1-194.7 (1996) ; Jakobovits A. Production of fully human antibodies by transgenic mice. Current Opinion in Biotechnology Vol. 6, No. 5, pp: 561-566 (1995) ; Mendez M, Abderrahim H, Noguchi M, David N, Hardy M, Green L, Tsuda H, Yoast S, Maynard-Currie C, Garza D, Gemmill R, Jakobovits A, Klapholz S. Analysis of the structural integrity of YACs comprising human immunoglobulin genes in yeast and in embryonic stem cells. Genomics Vol. 26, pp: 294-307 (1995); Jakobovits A. YAC Vectors: Humanizing the mouse genome. Current Biology Vol. 4, No. 8, pp: 761-763 (1994); Arbones M, Ord D, Ley K, Ratech H, Maynard-Curry K, Otten G, Capon D, Tedder T. Lymphocyte homing and leukocyte rolling and migration are impaired in L-selectin-deficient mice. Immunity Vol. 1, No. 4, pp: 247-260 (1994); Green L, Hardy M, Maynard-Curry K, Tsuda H, Louie D, Mendez M, Abderrahim H, Noguchi M, Smith D, Zeng Y, et. al. Antigen-specific human monoclonal antibodies from mice engineered with human Ig heavy and light chain YACs. Nature Genetics Vol. 7, No. 1, pp: 13-21 (1994); Jakobovits A, Moore A, Green L, Vergara G, Maynard-Curry K, Austin H, Klapholz S. Germ-line transmission and expression of a human-derived yeast artificial chromosome. Nature Vol. 362, No. 6417, pp: 255-258 (1993) ; Jakobovits A, Vergara G, Kennedy J, Hales J, McGuinness R, Casentini-Borocz D, Brenner D, Otten G. Analysis of homozygous mutant chimeric mice: deletion of the immunoglobulin heavy-chain joining region blocks B-cell development and antibody production. Proceedings of the National Academy of Sciences USA Vol. 90, No. 6, pp: 2551-2555 (1993); Kucherlapati et al., U.S. Pat. No. 6,1075,181.

Antibodies can also be made using phage display techniques. Such techniques can be used to isolate an initial antibody or to generate variants with altered specificity or avidity characteristics. Single chain Fv can also be used as is convenient. They can be made from vaccinated transgenic mice, if desired. Antibodies can be produced in cell culture, in phage, or in various animals, including but not limited to cows, rabbits, goats, mice, rats, hamsters, guinea pigs, sheep, dogs, cats, monkeys, chimpanzees, apes.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing antitumor agents (e.g., antisense oligonucleotides, plasmids which encode for toxins, methotrexate, etc.); and other antibodies or antibody fragments, such as F(ab).

Those of skill in the art will readily understand and be able to make such antibody derivatives, as they are well known in the art. The antibodies may be cytotoxic on their own, or they may be used to deliver cytotoxic agents to particular locations in the body. The antibodies can be administered to individuals in need thereof as a form of passive immunization.

Characterization of extracellular regions for the cell surface and secreted proteins from the protein sequence is based on the prediction of signal sequence, transmembrane domains and functional domains. Antibodies are preferably specifically immunoreactive with membrane associated proteins, particularly to extracellular domains of such proteins or to secreted proteins. Such targets are readily accessible to antibodies, which typically do not have access to the interior of cells or nuclei. However, in some applications, antibodies directed to intracellular proteins or epitopes may be useful as well. Moreover, for diagnostic purposes, an intracellular protein or epitope may be an equally good target since cell lysates may be used rather than a whole cell assay.

Computer programs can be used to identify extracellular domains of proteins whose sequences are known. Such programs include SMART software (Schultz et al., Proc. Natl. Acad. Sci. USA 95: 5857-5864, 1998) and Pfam software (Bateman et al., Nucleic acids Res. 28: 263-266, 2000) as well as PSORTII. Typically such programs identify transmembrane domains; the extracellular domains are identified as immediately adjacent to the transmembrane domains. Prediction of extracellular regions and the signal cleavage sites are only approximate. It may have a margin of error + or −5 residues. Signal sequence can be predicted using three different methods (Nielsen et al, Protein Engineering 10: 1-6 ,1997, Jagla et. al, Bioinformatics 16: 245-250 , 2000, Nakai, K and Horton, P. Trends in Biochem. Sci. 24:34-35, 1999) for greater accuracy. Similarly transmembrane (TM) domains can be identified by multiple prediction methods. (Pasquier, et. al, Protein Eng. 12:381-385, 1999, Sonnhammer et al., In Proc. of Sixth Int. Conf. on Intelligent Systems for Molecular Biology, p. 175-182 , Ed J. Glasgow, T. Littlejohn, F. Major, R. Lathrop, D. Sankoff, and C. Sensen Menlo Park, Calif.: AAAI Press, 1998 , Klein, et.al, Biochim. Biophys. Acta, 815:468, 1985, Nakai and Kanehisa Genomics, 14: 897-911, 1992). In ambiguous cases, locations of functional domains in well characterized proteins are used as a guide to assign a cellular localization.

Putative functions or functional domains of novel proteins can be inferred from homologous regions in the database identified by BLAST searches (Altschul et. al. Nucleic Acid Res. 25: 3389-3402, 1997) and/or from a conserved domain database such as Pfam (Bateman et.al, Nucleic Acids Res. 27:260-262 1999) BLOCKS (Henikoff, et. al, Nucl. Acids Res. 28:228-230, 2000) and SMART (Ponting, et. al, Nucleic Acid Res. 27,229-232, 1999). Extracellular domains include regions adjacent to a transmembrane domain in a single transmembrane domain protein (out-in or type I class). For multiple transmembrane domains proteins, the extracellular domain also includes those regions between two adjacent transmembrane domains (in-out and out-in). For type II transmembrane domain proteins, for which the N-terminal region is cytoplasmic, regions following the transmembrane domain is generally extracellular. Secreted proteins on the other hand do not have a transmembrane domain and hence the whole protein is considered as extracellular.

Membrane associated proteins can be engineered using standard techniques to delete the transmembrane domains, thus leaving the extracellular portions which can bind to ligands. Such soluble forms of transmembrane receptor proteins can be used to compete with natural forms for binding to ligand. Thus such soluble forms act as inhibitors. and can be used therapeutically as anti-angiogenic agents, as diagnostic tools for the quantification of natural ligands, and in assays for the identification of small molecules which modulate or mimic the activity of a TEM:ligand complex.

Alternatively, the endothelial markers themselves can be used as vaccines to raise an immune response in the vaccinated animal or human. For such uses, a protein, or immunogenic fragment of such protein, corresponding to the intracellular, extracellular or secreted TEM of interest is administered to a subject. The immogenic agent may be provided as a purified preparation or in an appropriately expressing cell. The administration may be direct, by the delivery of the immunogenic agent to the subject, or indirect, through the delivery of a nucleic acid encoding the immunogenic agent under conditions resulting in the expression of the immunogenic agent of interest in the subject. The TEM of interest may be delivered in an expressing cell, such as a purified population of tumor endothelial cells or a populations of fused tumor endothelial and dendritic cells. Nucleic acids encoding the TEM of interest may be delivered in a viral or non-viral delivery vector or vehicle. Non-human sequences encoding the human TEM of interest or other mammalian homolog can be used to induce the desired immunologic response in a human subject. For several of the TEMs of the present invention, mouse, rat or other ortholog sequences can be obtained from the literature or using techniques well within the skill of the art.

Endothelial cells can be identified using the markers which are disclosed herein as being endothelial cell specific. These include the 76 human markers identified herein, i.e., the tumor endothelial markers. Antibodies specific for such markers can be used to identify such cells, by contacting the antibodies with a population of cells containing some endothelial cells. The presence of cross-reactive material with the antibodies identifies particular cells as endothelial. Similarly, lysates of cells can be tested for the presence of cross-reactive material. Any known format or technique for detecting cross-reactive material can be used including, immunoblots, radioimmunoassay, ELISA, immunoprecipitation, and immunohistochemistry. In addition, nucleic acid probes for these markers can also be used to identify endothelial cells. Any hybridization technique known in the art including Northern blotting, RT-PCR, microarray hybridization, and in situ hybridization can be used.

One can identify tumor endothelial cells for diagnostic purposes, testing cells suspected of containing one or more TEMs. One can test both tissues and bodily fluids of a subject. For example, one can test a patient's blood for evidence of intracellular and membrane associated TEMs, as well as for secreted TEMs. Intracellular and/or membrane associated TEMs may be present in bodily fluids as the result of high levels of expression of these factors and/or through lysis of cells expressing the TEMs.

Populations of various types of endothelial cells can also be made using the antibodies to endothelial markers of the invention. The antibodies can be used to purify cell populations according to any technique known in the art, including but not limited to fluorescence activated cell sorting. Such techniques permit the isolation of populations which are at least 50, 60, 70, 80, 90, 92, 94, 95, 96, 97, 98, and even 99% the type of endothelial cell desired, whether normal, tumor, or pan-endothelial. Antibodies can be used to both positively select and negatively select such populations. Preferably at least 1, 5, 10, 15, 20, or 25 of the appropriate markers are expressed by the endothelial cell population.

Populations of endothelial cells made as described herein, can be used for screening drugs to identify those suitable for inhibiting the growth of tumors by virtue of inhibiting the growth of the tumor vasculature.

Populations of endothelial cells made as described herein, can be used for screening candidate drugs to identify those suitable for modulating angiogenesis, such as for inhibiting the growth of tumors by virtue of inhibiting the growth of endothelial cells, such as inhibiting the growth of the tumor or other undesired vasculature, or alternatively, to promote the growth of endothelial cells and thus stimulate the growth of new or additional large vessel or microvasculature.

Inhibiting the growth of endothelial cells means either regression of vasculature which is already present, or the slowing or the absence of the development of new vascularization in a treated system as compared with a control system. By stimulating the growth of endothelial cells, one can influence development of new (neovascularization) or additional vasculature development (revascularization). A variety of model screen systems are available in which to test the angiogenic and/or anti-angiogenic properties of a given candidate drug. Typical tests involve assays measuring the endothelial cell response, such as proliferation, migration, differentiation and/or intracellular interaction of a given candidate drug. By such tests, one can study the signals and effects of the test stimuli. Some common screens involve measurement of the inhibition of heparanase, endothelial tube formation on Matrigel, scratch induced motility of endothelial cells, platelet-derived growth factor driven proliferation of vascular smooth muscle cells, and the rat aortic ring assay (which provides an advantage of capillary formation rather than just one cell type).

Drugs can be screened for the ability to mimic or modulate, inhibit or stimulate, growth of tumor endothelium cells and/or normal endothelial cells. Drugs can be screened for the ability to inhibit tumor endothelium growth but not normal endothelium growth or survival. Similarly, human cell populations, such as normal endothelium populations or tumor endothelial cell populations, can be contacted with test substances and the expression of tumor endothelial markers determined. Test substances which decrease the expression of tumor endothelial markers (TEMs) are candidates for inhibiting angiogenesis and the growth of tumors. In cases where the activity of a TEM is known, agents can be screened for their ability to decrease or increase the activity.

Drug candidates capable of binding to TEM receptors found at the cell surface can be identified. For some applications, the identification of drug candidates capable of blocking the TEM receptor from its native ligand will be desired. For some applications, the identification of a drug candidate capable of binding to the TEM receptor may be used as a means to deliver a therapeutic or diagnostic agent. For other applications, the identification of drug candidates capable of mimicking the activity of the native ligand will be desired. Thus, by manipulating the binding of a transmembrane TEM receptor:ligand complex, one may be able to promote or inhibit further development of endothelial cells and hence, vascularization.

Expression can be monitored according to any convenient method. Protein or mRNA can be monitored. Any technique known in the art for monitoring specific genes' expression can be used, including but not limited to ELISAs, SAGE, microarray hybridization, Western blots. Changes in expression of a single marker may be used as a criterion for significant effect as a potential pro-angiogenic, anti-angiogenic or anti-tumor agent. However, it also may be desirable to screen for test substances which are able to modulate the expression of at least 5, 10, 15, or 20 of the relevant markers, such as the tumor or normal endothelial markers. Inhibition of TEM protein activity can also be used as a drug screen. Human and mouse TEMS can be used for this purpose.

Test substances for screening can come from any source. They can be libraries of natural products, combinatorial chemical libraries, biological products made by recombinant libraries, etc. The source of the test substances is not critical to the invention. The present invention provides means for screening compounds and compositions which may previously have been overlooked in other screening schemes. Nucleic acids and the corresponding encoded proteins of the markers of the present invention can be used therapeutically in a variety of modes. TEMs can be used to stimulate the growth of vasculature, such as for wound healing or to circumvent a blocked vessel. The nucleic acids and encoded proteins can be administered by any means known in the art. Such methods include, using liposomes, nanospheres, viral vectors, non-viral vectors comprising polycations, etc. Suitable viral vectors include adenovirus, retroviruses, and sindbis virus. Administration modes can be any known in the art, including parenteral, intravenous, intramuscular, intraperitoneal, topical, intranasal, intrarectal, intrabronchial, etc.

Specific biological antagonists of TEMs can also be used to therapeutic benefit. For example, antibodies, T cells specific for a TEM, antisense to a TEM, and ribozymes specific for a TEM can be used to restrict, inhibit, reduce, and/or diminish tumor or other abnormal or undesirable vasculature growth. Such antagonists can be administered as is known in the art for these classes of antagonists generally. Anti-angiogenic drugs and agents can be used to inhibit tumor growth, as well as to treat diabetic retinopathy, rheumatoid arthritis, psoriasis, polycystic kidney disease (PKD), and other diseases requiring angiogenesis for their pathologies.

The disclosure of co-pending application Ser. No. 09/918,715 is expressly incorporated herein.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Visualization of Vasculature of Colorectal Cancers

The endothelium of human colorectal cancer was chosen to address the issues of tumor angiogenesis, based on the high incidence, relatively slow growth, and resistance to anti-neoplastic agents of these cancers. While certain less common tumor types, such as glioblastomas, are highly vascularized and are regarded as good targets for anti-angiogenic therapy, the importance of angiogenesis for the growth of human colorectal cancers and other common solid tumor types is less well documented.

We began by staining vessels in colorectal cancers using von Willebrand Factor (vWF) as a marker. In each of 6 colorectal tumors, this examination revealed a high density of vessels throughout the tumor parenchyma. Interestingly, these analyses also substantiated the importance of these vessels for tumor growth, as endothelium was often surrounded by a perivascular cuff of viable cells, with a ring of necrotic cells evident at the periphery. Although these preliminary studies suggested that colon tumors are angiogenesis-dependent, reliable markers that could distinguish vessels in colon cancers from the vessels in normal colon are currently lacking One way to determine if such markers exist is by analyzing gene expression profiles in endothelium derived from normal and neoplastic tissue.

### EXAMPLE 2

### Purification of Endothelial Cells

Global systematic analysis of gene expression in tumor and normal endothelium has been hampered by at least three experimental obstacles. First, endothelium is enmeshed in a complex tissue consisting of vessel wall components, stromal cells, and neoplastic cells, requiring highly selective means of purifying ECs for analysis. Second, techniques for defining global gene expression profiles were not available until recently. And third, only a small fraction of the cells within a tumor are endothelial, mandating the development of methods that are suitable for the analysis of global expression profiles from relatively few cells.

To overcome the first obstacle, we initially attempted to purify ECs from dispersed human colorectal tissue using CD31, an endothelial marker commonly used for this purpose. This resulted in a substantial enrichment of ECs but also resulted in contamination of the preparations by hematopoietic cells, most likely due to expression of CD31 by macrophages. We therefore developed a new method for purifying ECs from human tissues using P1H12, a recently described marker for ECs. Unlike CD31, P1H12 was specifically expressed on the ECs of both colorectal tumors and normal colorectal mucosa. Moreover, immunofluorescence staining of normal and cancerous colon with a panel of known cell surface endothelial markers (e.g. VE-cadherin, CD31 and CD34) revealed that P1H12 was unique in that it stained all vessels including microvessels. In addition to selection with P1H12, it was necessary to optimize the detachment of ECs from their neighbors without destroying their cell surface proteins as well as to employ positive and negative affinity purifications using a cocktail of antibodies. The ECs purified from normal colorectal mucosa and colorectal cancers were essentially free of epithelial and hematopoietic cells as judged by RT-PCR and subsequent gene expression analysis (see below).

### EXAMPLE 3

### Comparison of Tumor and Normal Endothelial Cell Expression Patterns

To overcome the remaining obstacles, a modification of the Serial Analysis of Gene Expression (SAGE) technique was used. SAGE associates individual mRNA transcripts with 14 base pair tags derived from a specific position near their 3′ termini. The abundance of each tag provides a quantitative measure of the transcript level present within the mRNA population studied. SAGE is not dependent on pre-existing databases of expressed genes, and therefore provides an unbiased view of gene expression profiles. This feature is particularly important in the analysis of cells that constitute only a small fraction of the tissue under study, as transcripts from these cells are unlikely to be well represented in extant EST databases. We adapted the SAGE protocol so that it could be used on small numbers of purified ECs. A library of ˜100,000 tags from the purified ECs of a colorectal cancer, and a similar library from the ECs of normal colonic mucosa from the same patient were generated. These ˜193,000 tags corresponded to over 32,500 unique transcripts. Examination of the expression pattern of hematopoietic, epithelial and endothelial markers confirmed the purity of the preparations.

### EXAMPLE 4

### Tumor Versus Normal Endothelium

We next attempted to identify transcripts that were differentially expressed in endothelium derived from normal or neoplastic tissues. Forty-seven tags encoding transmembrane proteins were identified that were expressed at 2-fold or higher levels in tumor vessels. Those transcripts expressed at higher levels in tumor endothelium are most likely to be useful in the future for diagnostic and therapeutic purposes.

### REFERENCES AND NOTES

The disclosure of each reference cited is expressly incorporated herein.
1. J. Folkman, in Cancer Medicine J. Holland, Bast Jr, R C, Morton D L, Frei III, E, Kufe, D W, Weichselbaum, R R, Ed. (Williams & Wilkins, Baltimore, 1997) pp. 181.
2. R. S. Kerbel, Carcinogenesis 21, 505 (2000).
3. P. Wesseling, D. J. Ruiter, P. C. Burger, J Neurooncol 32, 253 (1997).
4. Q. G. Dong, et al., Arterioscler Thromb Vasc Biol 17, 1599 (1997).
5. P. W. Hewett, J. C. Murray, In Vitro Cell Dev Biol Anim 32, 462 (1996).
6. M. A. Hull, P. W. Hewett, J. L. Brough, C. J. Hawkey, Gastroenterology 111, 1230 (1996).
7. G. Haraldsen, et al., Gut 37, 225 (1995).
8. The original EC isolation protocol was the same as that shown in FIG. 2B except that dispersed cells were stained with anti-CD31 antibodies instead of anti-P1H12, and magnetic beads against CD64 and CD14 were not included in the negative selection. After generating 120,000 SAGE tags from these two EC preparations, careful analysis of the SAGE data revealed that, in addition to endothelial-specific markers, several macrophage-specific markers were also present.
9. A. Solovey, et al., N Engl J Med 337, 1584 (1997).
10. V. E. Velculescu, L. Zhang, B. Vogelstein, K. W. Kinzler, Science 270 , 484-487 (1995).
11. In order to reduce the minimum amount of starting material required from ˜50 million cells to ˜50,000 cells (i.e. ˜1000-fold less) we and others (38) have introduced several modifications to the original SAGE protocol. A detailed version of our modified “MicroSAGE” protocol is available from the authors upon request.
12. 96,694 and 96,588 SAGE tags were analyzed from normal and tumor derived ECs, respectively, and represented 50,298 unique tags. A conservative estimate of 32,703 unique transcripts was derived by considering only those tags observed more than once in the current data set or in the 134,000 transcripts previously identified in human transcriptomes (39).
13. To identify endothelial specific transcripts, we normalized the number of tags analyzed in each group to 100,000, and limited our analysis to transcripts that were expressed at levels at least 20-fold higher in ECs than in non-endothelial cell lines in culture and present at fewer than 5 copies per 100,000 transcripts in non-endothelial cell lines and the hematopoietic fraction (˜57,000 tags) (41). Non-endothelial cell lines consisted of 1.8×106 tags derived from a total of 14 different cancer cell lines including colon, breast, lung, and pancreatic cancers, as well as one non-transformed keratinocyte cell line, two kidney epithelial cell lines, and normal monocytes. A complete list of PEMs is available at www.sagenet.org\angio\table1.htm.
14. M. Tucci, et al., J Endocrinol 157, 13 (1998).
15. T. Oono, et al., J Invest Dermatol 100 , 329 (1993).
16. K. Motamed, Int J Biochem Cell Biol 31, 1363 (1999).
17. N. Bardin, et al., Tissue Antigens 48, 531 (1996).
18. D. M. Bradham, A. Igarashi, R. L. Potter, G. R. Grotendorst, J Cell Biol 114, 1285 (1991).
19. K. Akaogi, et al., Proc Natl Acad Sci USA 93, 8384 (1996).
20. Y. Muragaki, et al., Proc Natl Acad Sci USA 92, 8763 (1995).
21. M. L. Iruela-Arispe, C. A. Diglio, E. H. Sage, Arterioscler Thromb 11 , 805 (1991).
22. J. P. Girard, T. A. Springer, Immunity 2, 113 (1995).
23. E. A. Jaffe, et al., J Immunol 143, 3961 (1989).
24. J. P. Girard, et al., Am J Pathol 155, 2043 (1999).
25. H. Ohtani, N. Sasano, J Electron Microsc 36, 204 (1987).
26. For non-radioactive in situ hybridization, digoxigenin (DIG)-labelled sense and anti-sense riboprobes were generated through PCR by amplifying 500-600 by products and incorporating a T7 promoter into the anti-sense primer. In vitro transcription was performed using DIG RNA labelling reagents and T7 RNA polymerase (Roche, Indianapolis, Ind.). Frozen tissue sections were fixed with 4% paraformaldehyde, permeabilized with pepsin, and incubated with 200 ng/ml of riboprobe overnight at 55° C. For signal amplification, a horseradish peroxidase (HRP) rabbit anti-DIG antibody (DAKO, Carpinteria, Calif.) was used to catalyse the deposition of Biotin-Tyramide (from GenPoint kit, DAKO). Further amplification was achieved by adding HRP rabbit anti-biotin (DAKO), biotin-tyramide, and then alkaline-phosphatase (AP) rabbit anti-biotin (DAKO). Signal was detected using the AP substrate Fast Red TR/Napthol AS-MX (Sigma, St. Louis, Mo.), and cells were counterstained with hematoxylin unless otherwise indicated. A detailed protocol including the list of primers used to generate the probes can be obtained from the authors upon request.
27. Transcript copies per cell were calculated assuming an average cell contains 300,000 transcripts.
28. R. S. Warren, H. Yuan, M. R. Matli, N. A. Gillett, N. Ferrara, J Clin Invest 95, 1789 (1995).
29. Y. Takahashi, Y. Kitadai, C. D. Bucana, K. R. Cleary, L. M. Ellis, Cancer Res 55, 3964 (1995).
30. L. F. Brown, et al., Cancer Res 53, 4727 (1993).
31. Endothelial-specific transcripts were defined as those expressed at levels at least 5-fold higher in ECs in vivo than in non-endothelial cell lines in culture (13), and present at no more than 5 copies per 100,000 transcripts in non-endothelial cell lines and the hematopoietic cell fraction (41). Transcripts showing statistically different levels of expression (P<0.05) were then identified using Monte Carlo analysis as previously described (40). Transcripts preferentially expressed in normal endothelium were then defined as those expressed at levels at least 10-fold higher in normal endothelium than in tumor endothelium. Conversely, tumor endothelial transcripts were at least 10-fold higher in tumor versus normal endothelium. See www.sagenet.org\angio\table2.htm and www.sagenet.org\angio\table3.htm for a complete list of differentially expressed genes.
32. M. Iurlaro, et al., Eur J Clin Invest 29, 793 (1999).
33. W. S. Lee, et al., Circ Res 82, 845 (1998).
34. J. Niquet, A. Represa, Brain Res Dev Brain Res 95, 227 (1996).
35. L. Fouser, L. Iruela-Arispe, P. Bornstein, E. H. Sage, J Biol Chem 266 , 18345 (1991).
36. M. L. Iruela-Arispe, P. Hasselaar, H. Sage, Lab Invest 64, 174 (1991).
37. H. F. Dvorak, N Engl J Med 315, 1650 (1986).
38. B. Virlon, et al., Proc Natl Acad Sci USA 96, 15286 (1999).
39. V. E. Velculescu, et al., Nat Genet 23, 387 (1999).
40. L. Zhang, et al., Science 276, 1268 (1997).
41. Human colon tissues were obtained within ½ hour after surgical removal from patients. Sheets of epithelial cells were peeled away from normal tissues with a glass slide following treatment with 5 mM DDT, then 10 mM EDTA, leaving the lamina propria intact. After a 2 h incubation in collagenase at 37° C., cells were filtered sequentially through 400 um, 100 um, 50 um and 25 um mesh, and spun through a 30% pre-formed Percoll gradient to pellet RBCs. Epithelial cells (Epithelial Fraction), which were found to non-specifically bind magnetic beads, were removed using Dynabeads coupled to BerEP4 (Dynal, Lake Success, N.Y.). Subsequently, macrophages and other leukocytes (Hematopoietic Fraction) were removed using a cocktail of beads coupled to anti-CD45, anti-CD14 and anti-CD64 (Dynal). The remaining cells were stained with P1H12 antibody, purified with anti-mouse IgG-coupled magnetic beads, and lysed in mRNA lysis buffer. A detailed protocol can be obtained from the authors upon request.
42. H. Sheikh, H. Yarwood, A. Ashworth, C. M. Isacke, J Cell Sci 113, 1021-32 (2000).

TABLE 1
Membrane-associated tumor endothelial markers

| Unigene | | | Signal | | | | SEQ ID |
|---|---|---|---|---|---|---|---|
| ID | Function | OMIMID | Seq | Protein | TM Location | Orientation | NO |
| Hs.102308 | potassium inwardly-rectifying channel, | 600935 | no | NP_004973 | 73-95, 156-178 | IN | |
| | subfamily J, member 8 | | | | | | |
| Hs.109225 | Vascular cell adhesion molecule 1 | 192225 | yes | NP_001069 | 699-721 | Unsure | |
| Hs.110024 | NADH: ubiquinone oxidoreductase | | yes | NP_064527 | 20-42 | Unsure | |
| | MLRQ subunit homolog | | | | | | |
| Hs.125036 | TEM17 | 606826 | yes | NP_065138 | 425-447 | OUT | |
| Hs.125359 | TEM13, Thy-1 cell surface antigen | 188230 | yes | NP_006279 | 140-161 | Unsure | |
| Hs.13662 | hypothetical protein MGC5508 | | yes | NP_076997 | 84-106, 130-152, 159-176, | Unsure | |
| | | | | | 186-205 | | |
| Hs.1501 | syndecan 2 (heparan sulfate | 142460 | yes | AAA52701 | 147-169 | Unsure | |
| | proteoglycan 1, cell surface-associated, | | | | | | |
| | fibroglycan) | | | | | | |
| Hs.150540 | hypothetical protein BC002942 | | yes | NP_149977 | 367-389, 314-336, 79-101, | Unsure | |
| | | | | | 256-278, 108-130, 401-423, | | |
| | | | | | 639-661, 131-152, 13-35, | | |
| | | | | | 226-248 | | |
| Hs.155071 | TEM44, hypothetical protein FLJ11190 | | no | NP_060824 | 121-143, 177-199 | Unsure | |
| Hs.16187 | uncharacterized hematopoietic | | no | NP_060937 | 232-254 | OUT | |
| | stem/progenitor cells protein MDS032 | | | | | | |
| Hs.166994 | FAT tumor suppressor homolog 1 | 600976 | yes | NP_005236 | 4181-4203 | Unsure | |
| | (Drosophila) | | | | | | |
| Hs.17170 | G protein-coupled receptor 4 | 600551 | no | NP_005273 | 55-77, 92-113, 20-42, 225-244, | OUT | |
| | | | | | 183-205 | | |
| Hs.17270 | TEM9 | 606823 | yes | NP_116166 | 921-943, 764-786, 1041-1060, | Unsure | |
| | | | | | 878-900, 799-821, 1012-1034 | | |
| Hs.177486 | amyloid beta (A4) precursor protein | 104760 | yes | NP_000475 | 701-723 | Unsure | |
| | (protease nexin-II, Alzheimer disease) | | | | | | |
| Hs.180338 | tumor necrosis factor receptor | 603366 | yes | NP_683869 | 200-222 | IN | 9, 10 |
| | superfamily, formerly member 12, now | | | | | | |
| | member 25 (translocating chain- | | | | | | |
| | association membrane protein) | | | | | | |
| Hs.181244 | major histocompatibility complex, | 142800 | yes | NP_002107 | 305-327 | OUT | |
| | class I, A | | | | | | |
| Hs.185973 | degenerative spermatocyte homolog, | | yes | NP_003667 | 43-61, 160-177 | Unsure | |
| | lipid desaturase (Drosophila) | | | | | | |
| Hs.195727 | TEM1, endosialin | 606064 | yes | NP_065137 | 686-708 | Unsure | |
| Hs.198265 | matrix metalloproteinase 25 | | yes | NP_071913 | 541-562 | Unsure | |
| Hs.20166 | prostate stem cell antigen | 602470 | yes | NP_005663 | 100-122 | Unsure | |
| Hs.211579 | melanoma cell adhesion molecule | 155735 | yes | NP_006491 | 560-582 | OUT | |
| Hs.23016 | G protein-coupled receptor | | yes | | 47-69, 297-319, 82-104, | OUT | 3, 4 |
| | | | | | 214-236, 119-140, | | |
| | | | | | 160-182, 255-277 | | |
| Hs.231119 | protocadherin beta 9 | 606335 | yes | NP_061992 | 689-711, 13-35 | IN | |
| Hs.2399 | matrix metalloproteinase 14 (membrane- | 600754 | yes | NP_004986 | 540-562 | Unsure | |
| | inserted) | | | | | | |
| Hs.24220 | Scotin | 607290 | yes | NP_057563 | 110-132 | OUT | |
| Hs.24395 | chemokine (C-X-C motif) ligand 14 | 604186 | no | NP_004878 | 31-Oct | OUT | |
| Hs.251385 | murine retrovirus integration site 1 | 604673 | no | NP_569056 | 830-852 | Unsure | |
| | homolog | | | | | | |
| Hs.256297 | integrin, alpha 11 | 604789 | yes | NP_036343 | 1143-1165 | OUT | |
| Hs.265827 | interferon, alpha-inducible protein (clone | 147572 | yes | NP_075011 | 5-24, 44-66 | IN | |
| | IFI-6-16) | | | | | | |
| Hs.274127 | CLST 11240 protein | | no | NP_057522 | 62-84, 30-47 | IN | |
| Hs.274368 | TEM42, MSTP032 rev str; | | no | NP_079502 | 47-69 | OUT | 1, 2 |
| Hs.278568 | H factor (complement)-like 1 | 134371 | yes | NP_002104 | 23-Jan | Unsure | |
| Hs.27935 | tweety homolog 2 (Drosophila) | | yes | NP_116035 | 242-264, 89-111, 390-412, | IN | |
| | | | | | 215-237, 47-69 | | |
| Hs.279746 | transient receptor potential cation | 606676 | no | NP_057197 | 535-557, 391-413, 492-514, | Unsure | |
| | channel, subfamily V, member 2 | | | | 433-455, 622-644, 460-482 | | |
| Hs.283558 | Hypothetical protein PRO1855 | | no | NP_060979 | 246-268 | IN | |
| Hs.285814 | sprouty homolog 4 (Drosophila) | | no | AAK00653 | 236-258 | OUT | |
| Hs.291904 | accessory protein BAP31 | 300398 | yes | NP_005736 | 44-63, 102-121 | IN | |
| Hs.295726 | integrin, alpha V (vitronectin receptor, | 193210 | yes | NP_002201 | 994-1016 | OUT | |
| | alpha polypeptide, antigen CD51) | | | | | | |
| Hs.296310 | gap junction protein, alpha 4, 37 kDa | 121012 | no | NP_002051 | 207-229, 20-39, 76-98 | IN | |
| | (connexin 37) | | | | | | |
| Hs.29665 | calsyntenin 1 | | yes | NP_055759 | 860-882 | Unsure | |
| Hs.298476 | solute carrier family 26, member 6 | | no | NP_599025 | 380-402, 187-209, 115-137, | OUT | |
| | | | | | 475-506, 417-436, 264-283, | | |
| | | | | | 346-368, 141-163, 295-314, | | |
| | | | | | 443-460 | | |
| Hs.29882 | family with sequence similarity 3, | | yes | NP_055703 | 29-Jul | IN | |
| | member C | | | | | | |
| Hs.300697 | immunoglobulin heavy constant gamma | 147120 | yes | | 547-569 | OUT | |
| | 3 (G3m marker) | | | | | | |
| Hs.31720 | Hephaestin | 300167 | yes | NP_620074 | 1108-1130 | OUT | |
| Hs.322456 | Hypothetical protein DKFZp761D0211 | | no | NP_114428 | 49-71 | IN | |
| Hs.323769 | cisplatin resistance related protein | | yes | NP_110409 | 15-36, 401-423, 285-307, | IN | |
| | CRR9p | | | | 431-453, 345-362, 318-340 | | |
| Hs.324844 | Hypothetical protein IMAGE3455200 | | yes | NP_076869 | 75-97, 101-123, 116-138 | Unsure | |
| Hs.34665 | Homo sapiens mRNA full length insert | | no | | 456-478 | OUT | |
| | cDNA clone EUROIMAGE881791 | | | | | | |
| Hs.381200 | Hypothetical protein MGC15523 | | yes | NP_612637 | 378-397, 83-105, 120-142, | IN | |
| | | | | | 230-252, 323-340, 149-171, | | |
| | | | | | 344-366, 272-294, 36-58 | | |
| Hs.393 | Prostaglandin 12 (prostacyclin) receptor | 600022 | no | NP_000951 | 188-210, 49-71, 93-115, | OUT | |
| | (IP) | | | | 136-158, 238-260, 15-37 | | |
| Hs.43910 | CD164 antigen, sialomucin | 603356 | yes | NP_006007 | 164-186 | Unsure | |
| Hs.6459 | Putative G-protein coupled receptor | | yes | NP_078807 | 196-218, 46-68, 369-391, | IN | |
| | GPCR41 | | | | 81-103, 113-135, 404-426, | | |
| | | | | | 147-169, 325-347, 337-359, | | |
| | | | | | 9-31, 276-298 | | |
| Hs.7158 | DKFZP566H073 protein | | yes | NP_056343 | 172-194 | Unsure | |
| Hs.74615 | platelet-derived growth factor receptor, | 173490 | yes | NP_006197 | 527-549, 7-29 | IN | |
| | alpha polypeptide | | | | | | |
| Hs.74823 | NADH dehydrogenase (ubiquinone) 1 | 300078 | yes | NP_004532 | 27-May | OUT | |
| | alpha subcomplex, 1, 7.5 kDa | | | | | | |
| Hs.75564 | CD151 antigen | 602243 | yes | | 57-79, 92-114, 222-244 | IN | |
| Hs.76144 | platelet-derived growth factor receptor, | 173410 | yes | NP_002600 | 534-556 | Unsure | |
| | beta polypeptide | | | | | | |
| Hs.77665 | KIAA0102 gene product | | no | NP_055567 | 80-102, 112-134 | IN | |
| Hs.77873 | B7 homolog 3 | 605715 | yes | | 466-488 | IN | |
| Hs.7835 | TEM22, endocytic receptor (mannose | | yes | NP_006030 | 1412-1434 | OUT | |
| | receptor, C type 2); involved in cell-cell | | | | | | |
| | communication, cell adhesion | | | | | | |
| Hs.79410 | solute carrier family 4, anion exchanger, | 109280 | no | NP_003031 | 794-816, 1031-1053, 901-918, | OUT | |
| | member 2 (erythrocyte membrane | | | | 709-731, 988-1010, 752-774, | | |
| | protein band 3-like 1) | | | | 818-840, 931-950, 1114-1136, | | |
| | | | | | 1175-1197, 1188-1210, 1101-1123 | | |
| Hs.82002 | endothelin receptor type B | 131244 | yes | NP_000106 | 367-389, 104-126, 217-239, | Unsure | 5, 6 |
| | | | | | 138-160, 325-347, 175-197, | | |
| | | | | | 275-297 | | |
| Hs.82890 | defender against cell death 1 | 600243 | yes | NP_001335 | 29-51, 56-78, 93-112 | OUT | |
| Hs.83883 | transmembrane, prostate androgen | 606564 | yes | NP_064567 | 41-63 | OUT | |
| | induced RNA | | | | | | |
| Hs.8546 | Notch homolog 3 (Drosophila) | 600276 | yes | NP_000426 | 1641-1663, 1496-1518, 20-42 | Unsure | 7, 8 |
| Hs.890 | lymphotoxin beta (TNF superfamily, | 600978 | yes | NP_002332 | 21-43 | IN | |
| | member 3) | | | | | | |
| Hs.8966 | TEM19 var1 (long); cell-surface protein, | 606410 | yes | NP_115584 | 321-343 | IN | |
| | domain homology with leukointegrin | | | | | | |
| | (integrin alpha-D); ATR | | | | | | |
| Hs.9004 | chondroitin sulfate proteoglycan 4 | 601172 | yes | NP_001888 | 2224-2246 | Unsure | |
| | (melanoma-associated) | | | | | | |
| Hs.93765 | lipoma HMGIC fusion partner | 606710 | yes | NP_005771 | 87-109, 121-143, 12-34, | Unsure | |
| | | | | | 166-188 | | |
| Hs.95744 | hypothetical protein similar to ankyrin | | no | NP_061901 | 472-494, 289-311, 318-340, | OUT | |
| | repeat-containing priotein AKR1 | | | | 347-369, 374-395, 505-528 | | |
| Hs.17144 | short-chain dehydrogenase/reductase 1 | | yes | NP_004744 | | | |
| | SDR1 | | | | | | |
| Hs.32978 | proprotein convertase subtilisin/kexin | 604872 | yes | NP_004707 | | | |
| | type 7 PCSK7 | | | | | | |
| Hs.289770 | Homo sapiens mRNA; cDNA | | no | | | | |
| | DKF2p686D0720 | | | | | | |
| Hs.418 | fibroblast activation protein, alpha FAP | 600403 | yes | NP_004451 | | | |
| Hs.211579 | melanoma cell adhesion molecule | 155735 | yes | NP_006491 | | | |
| | mCAM | | | | | | |
| Hs.9383 | cystein-rich with EFG-like domains 1 | 607170 | yes | NP_056328 | | | |
| | CRELD1 | | | | | | |

1. An isolated molecule comprising an antibody variable region which specifically binds to an extracellular domain of a TEM protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1.

2. The molecule of claim 1 which is an intact antibody molecule.

3. The molecule of claim 1 which is a single chain variable region (ScFv).

4. The molecule of claim 1 which is a humanized antibody.

5. The molecule of claim 1 which is a human antibody.

6. The molecule of claim 1 which is bound to a cytotoxic moiety.

7. The molecule of claim 1 which is bound to a therapeutic moiety.

8. The molecule of claim 1 which is bound to a detectable moiety.

9. The molecule of claim 1 which is bound to an anti-tumor agent.

10. A method of inhibiting neoangiogenesis comprising:
administering to a subject in need thereof an effective amount of an isolated molecule comprising an antibody variable region which specifically binds to an extracellular domain of a TEM protein selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1, whereby neoangiogenesis is inhibited.

11. The method of claim 10 wherein the subject bears a vascularized tumor.

12. The method of claim 10 wherein the subject has polycystic kidney disease.

13. The method of claim 10 wherein the subject has diabetic retinopathy.

14. The method of claim 10 wherein the subject has rheumatoid arthritis.

15. The method of claim 10 wherein the subject has psoriasis.

16. A method for inhibiting tumor growth in a subject bearing a tumor, comprising:
administering to the subject an effective amount of an isolated molecule comprising an antibody variable region which specifically binds to an extracellular domain of a TEM protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD 151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1, whereby the growth of the tumor is consequently inhibited.

17-27. (canceled)

28. A soluble form of a human transmembrane protein, said soluble form lacking a transmembrane domain, said human transmembrane protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1.

29. The soluble form of claim 28 that consists of an extracellular domain of the human transmembrane protein.

30. A method of inhibiting neoangiogenesis in a patient in need thereof, comprising:
administering to the patient a soluble form of a human transmembrane protein, whereby neoangiogenesis in the patient is inhibited, said soluble form lacking a transmembrane domain, said human transmembrane protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1.

31. The method of claim 30 wherein the patient has a vascularized tumor.

32. The method of claim 30 wherein the patient has polycystic kidney disease.

33. The method of claim 30 wherein the patient has diabetic retinopathy.

34. The method of claim 30 wherein the patient has rheumatoid arthritis.

35. The method of claim 30 wherein the patient has psoriasis.

36. A method of identifying regions of neoangiogenesis in a patient, comprising:
administering to a patient a molecule comprising an antibody variable region which specifically binds to an extracellular domain of a protein, wherein said molecule is bound to a detectable moiety, said protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1;
detecting the molecule bound to the detectable moiety in the patient, thereby identifying regions of neoangiogenesis in the patient.

37. A method of screening for neoangiogenesis in a patient, comprising:
contacting a body fluid collected from a patient with a molecule comprising an antibody variable region which specifically binds to an extracellular domain of a protein selected from the group consisting of: potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1;
detecting material in the body fluid that is cross-reactive with the molecule, wherein detection of cross-reactive material indicates neo-angiogenesis in the patient.

38-47. (canceled)

48. A method for identifying endothelial cells, comprising:
contacting a population of cells with one or more molecules comprising a variable region which binds specifically to a protein selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential ; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1;
detecting cells in the population which have bound to said molecules;
identifying cells which are bound to said one or more molecules as endothelial cells.

49. The method of claim 48 further comprising:
isolating cells which have bound to said one or more molecules from cells which have not bound.

50. The method of claim 48 wherein said one or more molecules are intact antibodies.

51. A method for identifying endothelial cells, comprising:
contacting cDNA or mRNA of a population of cells with one or more nucleic acid hybridization probes which are complementary to a cDNA or mRNA for a gene selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1;
detecting cDNA or mRNA which have specifically hybridized to said nucleic acid hybridization probes;
identifying cells whose nucleic acids specifically hybridized to said nucleic acid hybridization probes as endothelial cells.

52. A method for inducing an immune response to a TEM protein in a mammal, comprising:
administering to a human subject who has or is at risk of developing a tumor a TEM protein or a nucleic acid encoding a TEM protein, wherein the TEM protein is selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1; whereby a humoral or cellular immune response to the TEM protein is raised in the human subject.

53. The method of claim 52 further comprising:
administering to the human subject an immune adjuvant to augment the immune response.

54. A method for stimulating vascular proliferation comprising:
administering to a subject with a wound a TEM protein or nucleic acid encoding a TEM protein, wherein the TEM protein is selected from the group consisting of potassium inwardly-rectifying channel, subfamily J, member 8; vascular cell adhesion molecule 1; NADH:ubiquinone oxidoreductase MLRQ subunit homolog; hypothetical protein MGC5508; syndecan 2 (heparan sulfate proteoglycan 1, cell surface-associated, fibroglycan); hypothetical protein BC002942; uncharacterized hematopoietic; stem/progenitor cells protein MDS032; FAT tumor suppressor homolog 1 (Drosophila); G protein-coupled receptor 4; amyloid beta (A4) precursor protein (protease nexin-II, Alzheimer disease); tumor necrosis factor receptor superfamily, member 25 (translocating chain-association membrane protein); major histocompatibility complex, class I, A; degenerative spermatocyte homolog, lipid desaturase (Drosophila); matrix metalloproteinase 25; prostate stem cell antigen; melanoma cell; adhesion molecule; G protein-coupled receptor; protocadherin beta 9; matrix; metalloproteinase 14 (membrane-inserted); scotin; chemokine (C-X-C motif) ligand 14; murine retrovirus integration site 1 homolog; integrin, alpha 11; interferon, alpha-; inducible protein (clone IFI-6-16); CLST 11240 protein; H factor (complement)-like; tweety homolog 2 (Drosophila); transient receptor potential; cation channel, subfamily V, member 2; hypothetical protein PRO1855; sprouty homolog 4 (Drosophila); accessory protein BAP31; integrin, alpha V (vitronectin receptor, alpha polypeptide, antigen CD51); gap junction protein, alpha 4, 37 kDa (connexin 37); calsyntenin 1; solute carrier family 26, member 6; family with sequence similarity 3, member C; immunoglobulin heavy constant gamma 3 (G3m marker); hephaestin; hypothetical protein DKFZp761D0211; cisplatin resistance related protein CRR9p; hypothetical protein IMAGE3455200; Homo sapiens mRNA full length insert cDNA clone EUROIMAGE881791; hypothetical protein MGC15523; prostaglandin 12 (prostacyclin) receptor (IP); CD164 antigen, sialomucin; putative G-protein coupled receptor GPCR41; DKFZP566H073 protein; platelet-derived growth factor receptor, alpha polypeptide; NADH dehydrogenase (ubiquinone) 1 alpha subcomplex, 1, 7.5 kDa; CD151 antigen; platelet-derived growth factor receptor, beta polypeptide; KIAA0102 gene product; B7 homolog 3; solute carrier family 4, anion exchanger, member 2 (erythrocyte membrane protein band 3-like 1); endothelin receptor type B; defender against cell death 1; transmembrane, prostate androgen induced RNA; Notch homolog 3 (Drosophila); lymphotoxin beta (TNF superfamily, member 3) chondroitin sulfate proteoglycan 4 (melanoma-associated); lipoma HMGIC fusion partner; hypothetical protein similar to ankyrin repeat-containing protein AKR1; SDR1 short-chain dehydrogenase/reductase 1; PCSK7 proprotein convertase subtilisin/kexin type 7; Homo sapiens mRNA, cDNA DKFZp686D0720 (from clone DKFZp686D0720); FAP fibroblast activation protein, alpha; MCAM melanoma cell adhesion molecule; and CRELD1 cysteine-rich with EGF-like domains 1; whereby wound healing is promoted.
